(19) United States
(12) Patent Application Publication
(10) Pub. No.: US 2022/0223292 A1
(43) Pub. Date: Jul. 14, 2022

(54) SYSTEM AND METHOD FOR UTILIZING DIGITAL FORENSICS, ARTIFICIAL INTELLIGENCE, AND MACHINE LEARNING MODELS TO PREVENT SUICIDAL BEHAVIOR

(71) Applicant: Stop Soldier Suicide, LLC, Durham, NC (US)

(72) Inventors: Glenn Devitt, Durham, NC (US); Chris Ford, Durham, NC (US)

(73) Assignee: Stop Soldier Suicide, LLC, Durham, NC (US)

(21) Appl. No.: 17/571,968

(22) Filed: Jan. 10, 2022

Related U.S. Application Data
(60) Provisional application No. 63/136,418, filed on Jan. 12, 2021.

(51) Int. Cl. G16H 50/30

(52) U.S. Cl. CPC G16H 50/30

(57) ABSTRACT

A system for utilizing digital forensics, artificial intelligence, and machine learning models to prevent suicidal behavior is disclosed. In particular, the system extracts data from devices associated with individuals that have died by suicide. The system applies intelligence techniques to collect additional data from third-party data aggregators and to collect online identities associated with the deceased individuals. The system may process the data from the devices, the additional data, and the online identities into datasets to be combined into a data lake. By utilizing artificial intelligence and/or machine learning algorithms on the data lake, the system generates a model associated with predictive behavior and patterns correlated with suicide. The system identifies individuals having characteristics and/or behaviors correlated with the model, and initiates services to reduce the risk for suicide for such individuals. The effectiveness of such services may also be determined by the system.

### CROSS REFERENCE TO RELATED APPLICATION

This application claims priority to and the benefit of U.S. Provisional Patent Application Ser. No. 63/136,418, filed on Jan. 12, 2021, which is hereby incorporated by reference in its entirety.

### FIELD OF THE INVENTION

The present application relates to suicide prevention technologies, behavior modification technologies, artificial intelligence technologies, machine learning technologies, cloud-computing technologies, data analysis technologies, digital forensics technologies, and more particularly, to a system and method for utilizing digital forensics, artificial intelligence, and machine learning models to prevent suicidal behavior.

### BACKGROUND

In today's society, individuals are increasingly involved with high-stress and often traumatic situations. The increased incidence of such situations has led to a notable increase in destructive behaviors, suicide attempts, and completed suicides by individuals. Unfortunately, this notable increase in destructive behaviors, suicide attempts, and suicides is especially true for the brave men and women who currently serve in the military or are veterans of the military. Notably, suicidality is typically a volatile and fluctuating dynamic, where otherwise well-managed suicide risk states can be shattered in mere moments by situational triggers. Research suggests that more than seventy percent of suicidal individuals deliberate only an hour or less before acting on a suicidal impulse, and approximately twenty five percent deliberate for less than five minutes. This impulsivity is more pronounced among males and is exacerbated when firearms are readily accessible. In order to predict and combat such self-destructive and suicidal behavior, various intervention techniques, therapy sessions, self-help measures, and monitoring technologies have been utilized. Nevertheless, despite fifty plus years of concerted efforts to predict and combat suicide at the individual level, such efforts have largely failed. Some recent artificial intelligence and machine learning applications have provided some insights into the public-facing personas of suicide decedents, but neither of these technologies has been able to answer the central questions of where to find and how to reach suicidal individuals in the last minutes, hours, and/or days leading up to their deaths by suicide.

As a result, there remains room for substantial enhancements to existing technologies and processes and for the development of new technologies and processes to mitigate self-destructive behaviors and the risk of suicide. While currently existing technologies provide for various benefits, such technologies still come with various drawbacks and inefficiencies. For example, currently existing processes and technologies often do not detect or prevent situational triggers early enough to have meaningful impact. Additionally, while currently existing processes may have short-term effectiveness on a case-by-case basis, existing processes often fail to have lasting long-term suicide mitigation capabilities across a broad spectrum of individuals. Based on the foregoing, current technologies may be improved and enhanced so as to provide for more effective monitoring, greater quality data, faster detection of at-risk behaviors, enhanced suicide prevention protocols and procedures, more effective intervention processes, higher quality predictive capabilities, and more effective identification of at-risk individuals. Such enhancements and improvements to methodologies and technologies may provide for enhanced quality-of-life for at-risk individuals and enhanced suicide mitigation capabilities.

### SUMMARY

A system and accompanying methods for utilizing digital forensics, artificial intelligence, and machine learning models to prevent suicidal behavior and/or self-destructive behavior are disclosed. In particular, the system and methods significantly enhance strategic suicide prevention objectives, such as those set forth by the United States Special Operations Command (USSOCOM), through the generation and use of a digital forensics, artificial intelligence, and machine learning model designed to validate predictors of fatal suicide behaviors. Notably, the system and methods are capable of answering the question of where to find and how to reach suicidal individuals in the last minutes, hours, and days leading up to their deaths by suicide. To that end, the system and methods may collect and process digital data from a variety of types of devices, such as, but not limited to, smartphones, smartwatches, tablets, laptops, computers, sensors, cameras, and/or other devices of individuals, military branch service members, and veterans who have died by suicide. The collected and processed data may include, but is not limited to, social media posts, direct messages, connections with friends and/or acquaintances, information associated with active online accounts, search and website history, blog and/or forum posts, mobile application activity, text messages, instant messages, photos and/or video files, document files and downloads, gaming profiles and history, any other data, or a combination thereof. The system and methods may then include utilizing intelligence techniques, such as open-source intelligence techniques, to obtain additional data from third-party data aggregators and to obtain online identities of individuals of interest. Once the relevant data is obtained, the system and methods may process the data into large datasets, which may then be combined into a data lake through a digital forensic data export.

The system and methods may then include utilizing artificial intelligence and/or machine learning algorithms and/or entity extraction applications against the processed data lake to generate models of predictive behavior and patterns that are most highly correlated with service member and veteran suicide. Additionally, the system and methods may also include generating look-alike model user profiles for service members that may be at risk for taking their own lives within a certain period of time. Based on the analysis and processing conducted by utilizing the system and methods, the system and methods may utilize the findings to construct a hypothetical roadmap of the online footprint and digital activities of high-risk individuals (e.g. soldiers) at various moments of their lives, such as their final moments. The system and methods may validate hypotheses associated with the research findings through several processes. For example, the system and methods may conduct real-time monitoring of online sites and social media platforms most frequented by at-risk individuals (e.g. service members and veterans), such as during the preparation and/or pre-action stages of suicidality. In certain embodiments, the system and methods may focus on sites or other online resources and destinations that are frequented in the last hour (or other desired timeframe) of a suicidal individual's life. The system and methods may then employ mitigation services, such as strategic messaging to drive high-risk website visitors to the suicide prevention/mitigation platform provided by the system and/or to proxy sites provided by the platform that are utilized to facilitate suicide-risk triage. As the various mitigation services are rendered or at other designated times, the system and methods may also include determining the effectiveness of the rendered services. For example, the system and methods may determine the effectiveness of specific monitoring, messaging, and intervention strategies through suicide risk assessments for individuals acquired via the system and methods. The model(s) generated by utilizing the system and methods may demonstrate sensitivity and specificity for high-risk and low-risk levels for suicide as well.

The system and methods may also be utilized to identify the subset of individuals (e.g. active-duty service members) that are assessed at highest suicide risk (i.e. a threshold level of risk) through the evidence-based model demonstrating sensitivity and specificity for suicide risk. Once suicide risk is stabilized and/or managed, the system and methods may include employing the use of investigators or components of the system itself to work with each individual at-risk for suicide to reconstruct his online activities in the last hours and/or days before he attempted suicide or sought emergency assistance. Furthermore, the system and methods may employ the use of a software application that may be utilized by at-risk individuals while they are being monitored by the system. For example, an at-risk individual may load the software application onto their mobile device, which may be utilized to monitor the individual's activities and/or behavior for patterns of behavior that may be deemed by the system to be at-risk behavior, such as when compared to the model(s) and/or look-alike profiles. If an activity and/or behavior is detected by the application that is associated with risk of suicide or self-destructive behavior, the application may flag such a risk and initiate countermeasures to mitigate the risk of suicide and/or self-destructive behavior. For example, the application may initiate a digital video conference between a health professional and the at-risk individual to intervene in real-time to attempted to stabilize the risk. Over time, the model utilized to identify at-risk individuals may be updated as new data and new individuals are connected to the system. As a result, the model will become more robust and finely tuned as new and/or updated data arrives into the system. The continuously trained and updated model (and profiles) will allow for increased accuracy and enhanced capabilities in detecting at-risk individuals, initiating countermeasures, and determining the effectiveness of such countermeasures in mitigating risk. The system and methods may further incorporate reporting capabilities to provide determined insights from data analysis through reports, queries, dashboards, and/or digital visualizations, which may be rendered for viewing on a user interface of any device of the system. Based on at least the foregoing, the system and methods provide for superior detection of at-risk individuals, reduced errors, greater efficiencies, enhanced reporting and visualization capabilities, more robust data and analysis, and enhanced information. Such enhancements facilitate reduced suicidal risk, enhanced quality-of-life for at-risk users, improved interactions between intervenors and at-risk individuals, and improved morale.

In one embodiment, a system for utilizing digital forensics, artificial intelligence, and machine learning models to prevent suicidal behavior is provided. The system may include a memory that stores instructions and a processor that executes the instructions to perform various operations of the system. The system may perform an operation that includes accessing, obtaining, and extracting data from devices associated with a plurality of deceased individuals associated with suicide. The devices may include, but are not limited to, smartphones, mobile devices, laptops, tablets, computers, smartwatches, sensors, any type of device, or a combination thereof. Additionally, to further supplement the extracted data, the system may perform an operation that includes applying any number of intelligence techniques to collect additional data associated with the plurality of deceased individuals from third-party data aggregators and to collect online identities associated with the plurality of deceased individuals. The system may perform an operation that includes processing the data from the devices, the additional data, and the online identities into a plurality of datasets. The plurality of data sets may be combined by the system into a data lake. By utilizing artificial intelligence algorithms and/or machine learning algorithms, the system may perform an operation that includes generating a model associated with predictive behavior and/or patterns correlated with suicide. Using the generated model, the system may identify a subset of individuals from a group of monitored individuals that are at risk for suicide based on the individuals having behaviors and/or characteristics correlated with the model. In certain embodiments, the system may require a threshold correlation before identifying any such individual as belonging to the subset. The system may then proceed to perform an operation that includes initiating a service(s) for the individuals to reduce the risk for suicide for the individuals. Once a service has been initiated for the individual(s), the system may analyze and determine the effectiveness of such services in reducing the risk for suicide. Over time, the system may determine which services are more effective than others.

In another embodiment, a method for utilizing digital forensics, artificial intelligence, and machine learning models to prevent suicidal behavior is disclosed. The method may include a memory that stores instructions and a processor that executes the instructions to perform the functionality of the method. In particular, the method may include accessing, obtaining, and/or extracting data from devices associated with a plurality of deceased individuals associated with suicide. The devices may be devices utilized by such individuals, accessed by such individuals, associated with such individuals, or a combination thereof. Additionally, the method may include applying one or more intelligence techniques to collect additional data associated with the plurality of deceased individuals from a third-party data aggregator(s) and to collect online identities associated with the plurality of deceased individuals as well. Such additional data and online identities may be utilized to supplement the data extracted from the devices associated with the deceased individuals. The method may then include processing the data from the devices, the additional data, and the online identities into a plurality of datasets to be combined into a data lake. By utilizing artificial intelligence algorithms and/or machine learning algorithms on the data lake, a model associated with predictive behavior and patterns correlated with suicide may be generated by utilizing the method. The method may then include monitoring a plurality of living individuals and identifying a subset of such individuals based on the individuals having a behavior, characteristic, or both, correlated with the model. Furthermore, the method may include initiating a service for the subset of individuals to reduce the risk for suicide and/or self-destructive behavior.

According to yet another embodiment, a computer-readable device comprising instructions, which, when loaded and executed by a processor cause the processor to perform operations, the operations comprising: extracting data from devices associated with a plurality of deceased individuals associated with suicide; applying an intelligence technique to collect additional data associated with the plurality of deceased individuals from a third-party data aggregator and to collect online identities associated with the plurality of deceased individuals; processing the data from the devices, the additional data, and the online identities into a plurality of datasets to be combined into a data lake; generating, by utilizing an artificial intelligence algorithm on the data lake, a model associated with predictive behavior and patterns correlated with suicide; identifying an individual at risk for suicide based on the individual having a behavior, characteristic, or both, correlated with the model; and initiating a service for the individual to reduce the risk for suicide for the individual.

These and other features of the systems and methods for utilizing digital forensics, artificial intelligence, and machine learning models to prevent suicidal behavior are described in the following detailed description, drawings, and appended claims.

### BRIEF DESCRIPTION OF THE DRAWINGS

FIG. 1 is a schematic diagram of a system for utilizing digital forensics, artificial intelligence, and machine learning models to prevent suicidal behavior according to an embodiment of the present disclosure.

FIG. 2 is a chart illustrating aspects of digital forensic analysis for use with the system of FIG. 1.

FIG. 3 is a sample platform for use with the system of FIG. 1 that is configured to process and manage data of the system of FIG. 1.

FIG. 4 is a flow diagram illustrating a sample method for utilizing digital forensics, artificial intelligence, and machine learning models to prevent suicidal behavior according to an embodiment of the present disclosure.

FIG. 5 is a schematic diagram of a machine in the form of a computer system within which a set of instructions, when executed, may cause the machine to utilize digital forensics, artificial intelligence, and machine learning models to prevent suicidal behavior.

### DETAILED DESCRIPTION OF THE DRAWINGS

A system 100 and accompanying methods for utilizing digital forensics, artificial intelligence, and machine learning models to prevent suicidal behavior are disclosed. In particular, the system 100 and methods significantly enhance strategic suicide prevention objectives, such as those set forth by the USSOCOM, through the generation and use of a digital forensics, artificial intelligence, and machine learning model designed to validate predictors of fatal suicide behaviors. Notably, the functionality provided by the system 100 and methods facilitates the reaching of suicidal individuals in the last minutes, hours, and days leading up to their deaths by suicide. In order to do so, the system 100 and methods may collect and process data from a variety of types of devices, such as, but not limited to, smartphones, smartwatches, tablets, laptops, computers, sensors, cameras, and/or other devices of individuals, military branch service members, and veterans who have died by suicide. The collected and processed data may include, for example, social media posts, direct messages, information relating to connections with friends and/or acquaintances, information associated with active online accounts, search and website history, blog and/or forum posts, mobile application activity, text messages, instant messages, photos and/or video files, document files and downloads, gaming profiles and history, any other data, or a combination thereof. The system 100 and methods may then utilize intelligence techniques, such as open-source intelligence techniques, to obtain additional data from third-party data aggregators and to obtain online identities of individuals of interest. Once the relevant data is obtained, the system 100 and methods may process the data into large datasets, which may be combined into a data lake, such as through a digital forensic data export.

The system 100 and methods may then include utilizing artificial intelligence, machine learning algorithms and/or entity extraction applications on the processed data lake to generate models of predictive behavior and patterns that are most highly correlated with service member and veteran suicide. The system 100 and methods may also include generating model user profiles for service members that may be at risk for taking their own lives. Each model user profile may include different sets of characteristics that may have varying correlation with suicide and/or self-destructive behavior. The system 100 and methods may utilize the findings associated with the data to construct a hypothetical roadmap of the online footprint and digital activities of high-risk individuals at various moments of their lives. The system 100 and methods may validate hypotheses associated with the research findings through several processes. In particular, the system 100 and methods may conduct real-time monitoring of online sites and social media platforms most frequented by at-risk individuals (e.g. service members and veterans), such as during the preparation and/or pre-action stages of suicide. In certain embodiments, the system 100 and methods may focus on sites or other online resources that are frequented in the last hour (or other desired timeframe) of a suicidal individual's life. The system 100 and methods may then initiate mitigation services, such as strategic messaging to drive high-risk website visitors to the suicide prevention/mitigation platform provided by the system 100 and/or to proxy sites provided by the system 100 that are utilized to facilitate suicide risk triage. As the various mitigation services are rendered or at other designated times, the system 100 and methods may also include determining the effectiveness of the services. For example, the system 100 and methods may determine the effectiveness of specific monitoring, messaging, and intervention strategies through suicide risk assessments for individuals acquired via the system 100 and methods. The models generated by utilizing the system 100 and methods may demonstrate sensitivity and specificity for high-risk and low-risk levels (high risk and low risk may be indicated by threshold correlations and/or threshold ranges of correlations) for suicide as well.

The system 100 and methods may also be utilized to identify a subset of individuals (e.g. active-duty service members) that are assessed by the system 100 as having a highest suicide risk (i.e. a threshold level of risk) through the evidence-based model demonstrating sensitivity and specificity for suicide risk. Once suicide risk is stabilized and/or managed, the system 100 and methods may include employing the use of investigators or components of the system 100 itself to work with each at-risk individual to reconstruct his online activities in the last hours and/or days before he attempted suicide or sought emergency assistance. Additionally, the system 100 and methods may employ the use of a software application that may be utilized by the at-risk individuals while they are being monitored by the system 100. For example, an at-risk individual may execute the software application via his mobile device (e.g. first user device 102), which may be utilized to monitor the individual's activities and/or behavior. If an activity and/or behavior detected by the application is associated with risk of suicide or self-destructive behavior, the application may flag such activity and/or behavior and may initiate countermeasures to mitigate the risk of suicide and/or self-destructive behavior. Over time, the model utilized to identify at-risk individuals may be updated as new data and new individuals are input and connected to the system 100 respectively. As a result, the model becomes more robust and finely tuned as new and/or updated data arrives into the system 100 over time. The continuously trained and updated model will allow for increased accuracy and enhanced capabilities in detecting at-risk individuals, initiating countermeasures, and determining the effectiveness of such countermeasures in mitigating risk. The system 100 and methods may further incorporate reporting capabilities to provide determined insights from data analysis through reports, queries, dashboards, and/or digital visualizations. Based on at least the foregoing, the system 100 and methods provide for superior detection of at-risk individuals, reduced errors, greater efficiencies, enhanced reporting and visualization capabilities, more robust data and analysis, and enhanced information. Such enhancements facilitate reduced suicidal risk, enhanced quality-of-life for at-risk users, improved interactions between intervenors and at-risk individuals, and improved morale.

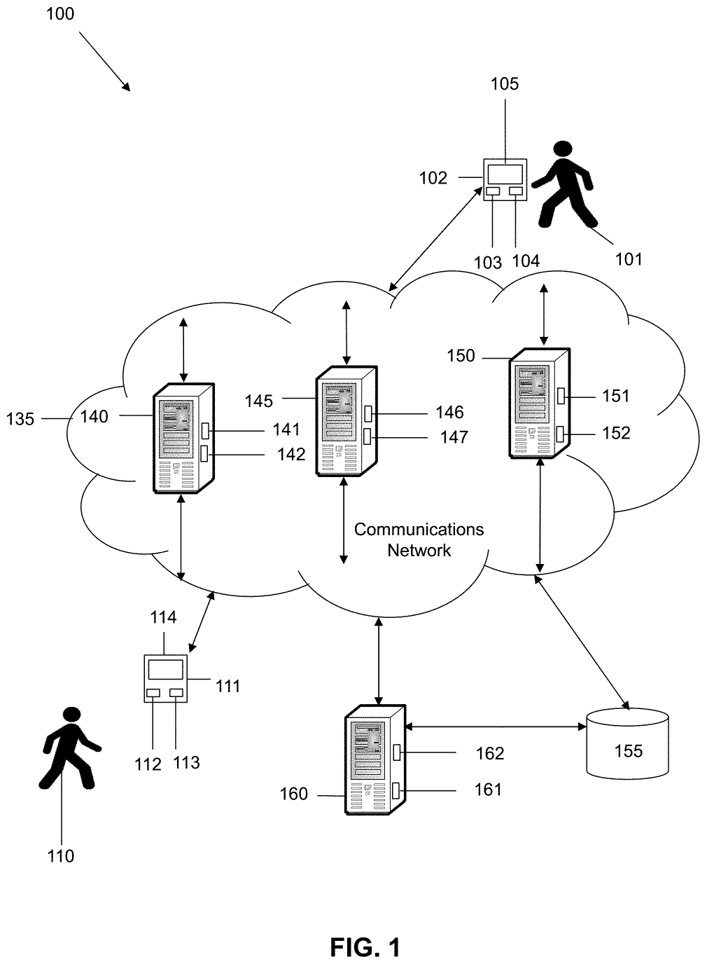

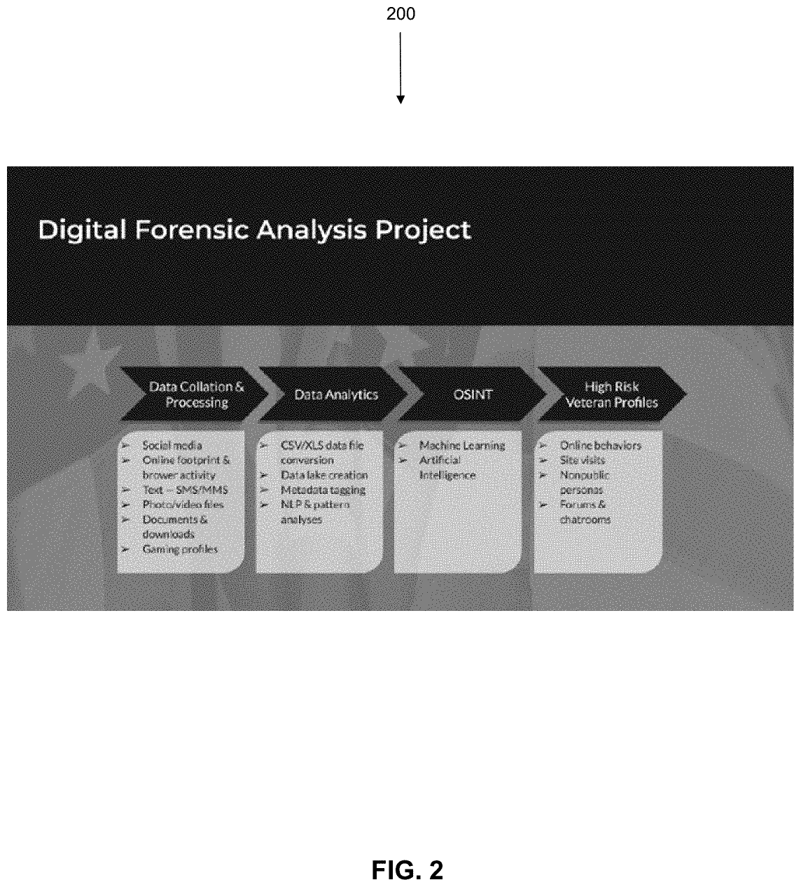

As shown in FIG. 1 and referring also to FIGS. 2-5, a system 100 utilizing digital forensics, artificial intelligence, and machine learning models to prevent suicidal behavior is disclosed. Notably, the system 100 may be configured to support, but is not limited to supporting, suicide prevention and/or intervention systems and services, alert systems and services, data analytics systems and services, data collation and processing systems and services, medical systems and services, artificial intelligence services and systems, machine learning services and systems, content delivery services, surveillance and monitoring services, cloud computing services, satellite services, telephone services, voice-over-internet protocol services (VoIP), software as a service (SaaS) applications, platform as a service (PaaS) applications, gaming applications and services, social media applications and services, operations management applications and services, productivity applications and services, mobile applications and services, and/or any other computing applications and services. Notably, the system 100 may include a first user 101, who may utilize a first user device 102 to access data, content, and services, or to perform a variety of other tasks and functions. As an example, the first user 101 may utilize first user device 102 to transmit signals to access various online services and content, such as those available on an internet, on other devices, and/or on various computing systems. As another example, the first user device 102 may be utilized to access an application that provides the operative functions of the system 100. In certain embodiments, the first user 101 may be a suicide prevention and/or reduction specialist, a suicidologist, a soldier, a physician, any type of person, a robot, a humanoid, a program, a computer, any type of user, or a combination thereof. The first user device 102 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, etc.) that may enable the first user 101 to interact with various applications executing on the first user device 102 and to interact with the system 100. In certain embodiments, the first user device 102 may be and/or may include a computer, any type of sensor, a laptop, a set-top-box, a tablet device, a phablet, a server, a mobile device, a smartphone, a smart watch, and/or any other type of computing device. Illustratively, the first user device 102 is shown as a smartphone device in FIG. 1. In certain embodiments, the first user device 102 may be utilized by the first user 101 to control and/or provide some or all of the operative functionality of the system 100.

In addition to using first user device 102, the first user 101 may also utilize and/or have access to additional user devices. As with first user device 102, the first user 101 may utilize the additional user devices to transmit signals to access various online services and content. The additional user devices may include memories that include instructions, and processors that executes the instructions from the memories to perform the various operations that are performed by the additional user devices. In certain embodiments, the processors of the additional user devices may be hardware, software, or a combination thereof. The additional user devices may also include interfaces that may enable the first user 101 to interact with various applications executing on the additional user devices and to interact with the system 100. In certain embodiments, the first user device 102 and/or the additional user devices may be and/or may include a computer, any type of sensor, a laptop, a set-top-box, a tablet device, a phablet, a server, a mobile device, a smartphone, a smart watch, and/or any other type of computing device, and/or any combination thereof. Sensors may include, but are not limited to, motion sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

The first user device 102 and/or additional user devices may belong to and/or form a communications network. In certain embodiments, the communications network may be a local, mesh, or other network that enables and/or facilitates various aspects of the functionality of the system 100. In certain embodiments, the communications network may be formed between the first user device 102 and additional user devices through the use of any type of wireless or other protocol and/or technology. For example, user devices may communicate with one another in the communications network by utilizing any protocol and/or wireless technology, satellite, fiber, or any combination thereof. Notably, the communications network may be configured to communicatively link with and/or communicate with any other network of the system 100 and/or outside the system 100.

In certain embodiments, the first user device 102 and additional user devices belonging to the communications network may share and exchange data with each other via the communications network. For example, the user devices may share information relating to the various components of the user devices, information associated with images and/or content accessed by a user of the user devices, information associated with content associated with suicide and/or self-destructive behavior, information associated with services for mitigating the risk of suicide, information identifying the locations of the user devices, information indicating the types of sensors that are contained in and/or on the user devices, information identifying the applications being utilized on the user devices, information identifying how the user devices are being utilized by a user, information identifying user profiles for users of the user devices, information identifying device profiles for the user devices, information identifying the number of devices in the communications network, information identifying devices being added to or removed from the communications network, any other information, or any combination thereof.

In addition to the first user 101, the system 100 may also include a second user 110. The second user 110 may be a person that may have self-destructive and/or suicidal behavior, a patient, a solider, military personnel, any type of user, or a combination thereof. In certain embodiments, the first user 101 may be a user that monitors the well-being of the second user 110 and/or is capable of providing services to the second user 110 to reduce the second user's 110 risk of self-destructive behavior and/or actions. The second user device 111 may be utilized by the second user 110 (or even potentially the first user 101) to transmit signals to request various types of content, services, and data provided by and/or accessible by communications network 135 or any other network in the system 100. In further embodiments, the second user 110 may be a robot, a computer, a humanoid, an animal, any type of user, or any combination thereof. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. screen, monitor, graphical user interface, etc.) that may enable the first user 101 to interact with various applications executing on the second user device 111 and to interact with the system 100. In certain embodiments, the second user device 111 may be a computer, a laptop, a set-top-box, a tablet device, a phablet, a server, a mobile device, a smartphone, a smart watch, and/or any other type of computing device. Illustratively, the second user device 111 is shown as a mobile device in FIG. 1. In certain embodiments, the second user device 111 may also include sensors, such as, but are not limited to, motion sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

In certain embodiments, the first user device 102, the additional user devices, and/or the second user device 111 may have any number of software applications and/or application services stored and/or accessible thereon. For example, the first user device 102, the additional user devices, and/or the second user device 111 may include applications for controlling the operative features and functionality of the system 100, applications for controlling any device of the system 100, suicide and/or self-destruction prevention applications, interactive social media applications, biometric applications, cloud-based applications, VoIP applications, other types of phone-based applications, product-ordering applications, business applications, e-commerce applications, media streaming applications, content-based applications, media-editing applications, database applications, gaming applications, internet-based applications, browser applications, mobile applications, service-based applications, productivity applications, video applications, music applications, social media applications, any other type of applications, any types of application services, or a combination thereof. In certain embodiments, the software applications may support the functionality provided by the system 100 and methods described in the present disclosure. In certain embodiments, the software applications and services may include one or more graphical user interfaces so as to enable the first and/or second users 101, 110 to readily interact with the software applications. The software applications and services may also be utilized by the first and/or second users 101, 110 to interact with any device in the system 100, any network in the system 100, or any combination thereof. In certain embodiments, the first user device 102, the additional user devices, and/or the second user device 111 may include associated telephone numbers, device identities, or any other identifiers to uniquely identify the first user device 102, the additional user devices, and/or the second user device 111.

The system 100 may also include a communications network 135. The communications network 135 may be under the control of a service provider, the first user 101, the second user 110, any other designated user, a computer, another network, or a combination thereof. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry. The communications network 135 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 140, 145, and 150 are shown as being included within communications network 135. In certain embodiments, the communications network 135 may be part of a single autonomous system that is located in a particular geographic region, or be part of multiple autonomous systems that span several geographic regions.

Notably, the functionality of the system 100 may be supported and executed by using any combination of the servers 140, 145, 150, and 160. The servers 140, 145, and 150 may reside in communications network 135, however, in certain embodiments, the servers 140, 145, 150 may reside outside communications network 135. The servers 140, 145, and 150 may provide and serve as a server service that performs the various operations and functions provided by the system 100. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 145 may include a memory 146 that includes instructions, and a processor 147 that executes the instructions from the memory 146 to perform the various operations that are performed by the server 145. Furthermore, the server 150 may include a memory 151 that includes instructions, and a processor 152 that executes the instructions from the memory 151 to perform the various operations that are performed by the server 150. In certain embodiments, the servers 140, 145, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 145, 150 may be communicatively linked to the communications network 135, any network, any device in the system 100, or any combination thereof.

The database 155 of the system 100 may be utilized to store and relay information that traverses the system 100, cache content that traverses the system 100, store data about each of the devices in the system 100 and perform any other typical functions of a database. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or be connected to a processor and memory to perform the various operation associated with the database 155. In certain embodiments, the database 155 may be connected to the servers 140, 145, 150, 160, the first user device 102, the second user device 111, the additional user devices, any devices in the system 100, any process of the system 100, any program of the system 100, any other device, any network, or any combination thereof.

The database 155 may also store information and metadata obtained from the system 100, store metadata and other information associated with the first and second users 101, 110, store data obtained from devices associated with deceased and/or living individuals, store online identities associated with deceased and/or living individuals, store information obtained from third-party data aggregators associated with deceased and/or living individuals, store models generated by the system 100, store information for updating the models, store information and protocols for mitigating suicide and/or self-destructive behavior, store information for use in services to mitigate suicide and/or self-destructive behavior, store information corresponding to interviews held with individuals, store information associated with behaviors and/or actions conducted by individuals, store user profiles associated with the first and second users 101 and/or having correlation with other users and/or deceased individuals, 110, store device profiles associated with any device in the system 100, store communications traversing the system 100, store user preferences, store information associated with any device or signal in the system 100, store information relating to patterns of usage relating to the user devices 102, 111, store any information obtained from any of the networks in the system 100, store historical data associated with the first and second users 101, 110, store device characteristics, store information relating to any devices associated with the first and second users 101, 110, store information associated with the communications network 135, store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

Operatively, the system 100 may operate and/or execute the functionality as described in the methods (e.g. method 400 as described below) of the present disclosure and in the following description. In particular, the system 100 may involve decedent device acquisition from deceased individuals, development of the predictive models as described herein, and the generation of look-alike profiles for service members (or other individuals) at risk for taking their lives. The system 100 also involves utilizing the data and analyses generated by the system 100 in combination with evidence-based suicide prevention and intervention processes to improve the effectiveness of existing suicide prevention protocols and procedures, such as, but not limited to, those provided by the U.S. Army and USSOCOM. Referring now to FIG. 2, an example process flow 200 for use with the system 100 is shown. The process flow 200 may be utilized to conduct forensic analyses and measures on data obtained by the system 100. Initially, the process flow 200 may include accessing and/or extracting data associated with deceased individuals, such as those that have died via suicide, from devices associated with such individuals. In certain embodiments, the data may be obtained from personal computers, smartwatches, mobile devices, sensors, laptops, tablets, and/or any other devices associated with such individuals. In certain embodiments, the data may include, but is not limited to, browser activity, digital online footprints, social media posts, direct messages, information associated with contacts of the deceased individuals, search history, website history, blog posts, forum posts, mobile application activity, text messaging (SMS/MMS/other messaging forms), photos, videos, audio, media content, documents, files, downloaded information, uploaded information, video game profiles, video game history, patterns of usage relating to use of the devices, sensor data (e.g. heart-rate data, sweat data, oxygen data, temperature data, pressure data, biometric data, and/or any other sensor data), any other data, or a combination thereof. In certain embodiments, the process flow 200 may also include gathering data from devices associated with living individuals (e.g. first and/or second users 101, 110), who may or may not have self-destructive behavior and/or risk for suicide. The obtained data may be collated and processed by the system 100.

Once the data is collected and/or as the data is being collected, the process flow 200 may conduct data analytics on the data. In certain embodiments, the process flow 200 may include performing CSV/XLS data file conversions (and/or other suitable conversions), generating datasets of the data for inclusion in a created data lake, conducting metadata tagging of the data, and/or conducting pattern analyses and/or natural language processing of the data. In certain embodiments, the metadata tagging may include, but is not limited to, tagging data to indicate a correlation with suicidal risk and/or self-destructive behavior. The metadata tagging may also tag data as not being correlated with suicidal risk and/or self-destructive behavior. In further embodiments, the level of suicidal risk and/or self-destructive behavior may also be indicated in the metadata associated with the data. For example, a scale of 0-100 may be utilized to indicate risk, where 100 is maximum risk and 0 is lowest risk. The metadata tagging may also include tagging the data with metadata indicating what type of data a particular piece of data is, tagging the data with what the data is and/or includes, tagging the form of the data, tagging the format of the data, tagging how relevant the data is with respect to suicidal risk and/or self-destructive behavior, tagging the data as being associated with and/or correlated with suicidal risk and/or self-destructive behavior, tagging whether data is related to other data and in what way, performing any other type of tagging, or a combination thereof. In addition to the metadata tagging, the system 100, such as via process flow 200, may also conduct natural language processing and/or pattern analyses on the data. The natural language processing and/or pattern analyses may be utilized to obtain meaning from the data, extract concepts from the data, detect patterns between and/or within data, determine the context associated with the data, obtain insights into the data, determine correlations between and/or among data (e.g. certain data is associated with suicidal behavior and/or self-destructive behavior), any other information from the data, or a combination thereof.

The process flow 200 may also include utilizing any number of intelligence techniques (e.g. Open-Source Intelligence Techniques (OSINT)) to collect data from third-party data aggregators and/or online identities associated with deceased individuals and/or living individuals. The collected data may be included by the system 100 within the generated data lake as well. The process flow 200 may then include utilizing artificial intelligence and/or machine learning algorithms on the data contained in the data lake to generate any number of models of predictive behavior and/or patterns correlated with suicidal behavior and/or self-destructive behavior. Each model may be file, program, module, and/or process that may be trained by the system 100 to recognize certain patterns, behaviors, and/or content correlated with suicide and/or self-destructive behavior (or other correlation of interest). The system 100 may train the model to learn from data fed into the system 100 so that the model may generate and/or facilitate the generation of predictions about new data and information that enters into the system 100. For example, as data is analyzed and/or processed by the system 100, certain images, websites, search history, sensor data, and/or other information may be determined by the system 100 to be associated with risk of suicide and/or self-destructive behavior. Such data may be tagged or otherwise marked as being associated with risk of suicide and/or self-destructive behavior for the model such that when new and/or subsequent data is analyzed by the system 100, the system 100 may utilize the model's information to determine whether the new data is associated with risk of suicide and/or self-destructive behavior.

The process flow 200 may also involve the creation of various types of profiles correlated with suicidal behavior, correlated with self-destructive behavior, and/or not correlated with such behavior. Each profile may have different characteristics associated with it. For example, a profile may have certain online behaviors associated with it, website history, non-public personas, forum and chatroom visits and/or posts, demographic information, psychographic information, gender information, education information, life experience information, health information, and/or other information. The set of information contained in each profile may cause the profile to have a certain correlation with suicidal risk and/or self-destructive behavior. In certain embodiments, the correlation may range from 0 to 1 or from 0 to 100 (or other desired scale) to show the correlation with suicidal risk and/or self-destructive behavior. The profiles and/or models may then be compared to behaviors and/or characteristics of monitored living individuals to determine whether the living individuals have a correlation to the profiles and/or models. If so, the process flow 200 may include identifying such living individuals as being at-risk for suicide and/or self-destructive behavior. The process flow 200 may then include initiating any number of services to mitigate the risk of suicide and/or self-destructive behavior for any such individuals. For example, the process flow 200 may include causing a signal to be sent to a device of an individual that interrupts the individual from continuing the self-destructive behavior and/or a suicide attempt. In certain embodiments, the process flow 200 may include redirecting the individual to a suicide prevention website, enabling the individual to receive audio and/or media content to persuade the individual away from the behavior, provide a link to a device of the individual to initiate a digital therapy session with a medical expert and/or therapist, enabling the individual to receive any type of interruption and/or service, or a combination thereof.

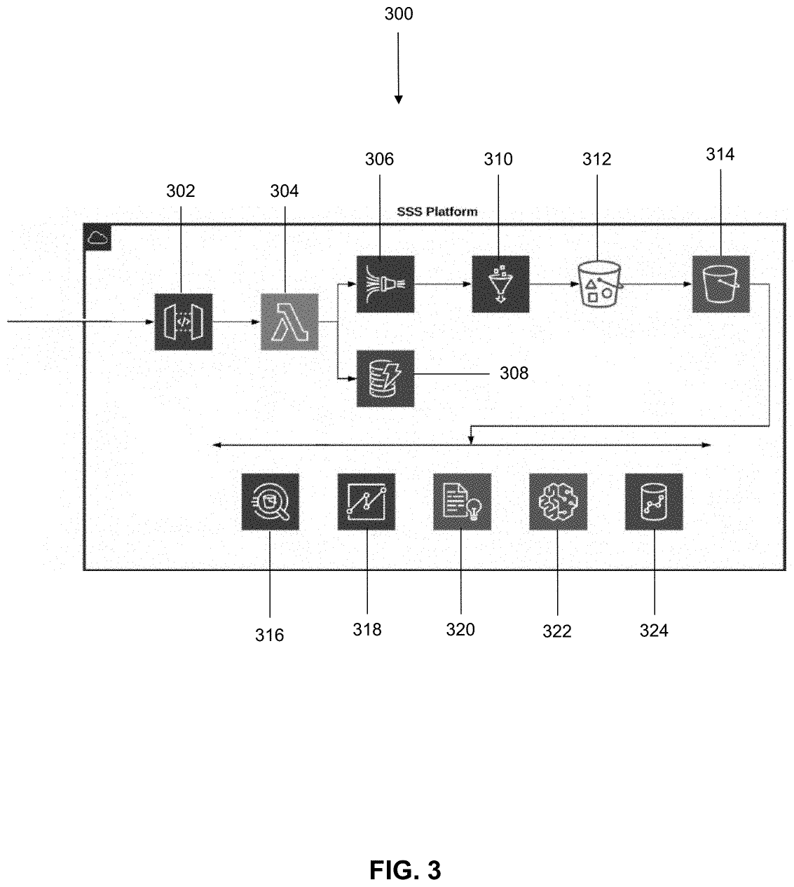

Notably, in certain embodiments and referring now also to FIG. 3, the platform 300 may be utilized to facilitate the performance of the steps of the process flow 200, the functionality of the system 100, and/or the steps of the method 400. In certain embodiments, the platform 300 does not have to be utilized to facilitate the functionality of the system 100 and/or steps of the method 400. The platform 300 may include, but is not limited to, a gateway 302 (e.g Amazon Gateway); a serverless computing platform 304 that runs code in response to events and manages computing resources (e.g. Amazon Lambda); a data firehose 306 for loading data into data lakes, data stores, and/or analytics services (e.g. Amazon Kinesis Data Firehose); a database/database service 308 (e.g. Amazon DynamoDB); a serverless ETL service that crawls through data, builds a data catalog, performs data preparation, and data ingestion (e.g. AWS Glue); an efficient data storage format for data processing formats (e.g. Apache Parquet); a service for providing object storage through a web service interface (e.g. Amazon Simple Storage Service); a service for conductive serverless interactive query services to query data and analyze big data (e.g. Amazon Athena); a machine learning-powered intelligence service that allows for the creation and publishing of interactive dashboards to output and illustrate insights associated with analyzed data (e.g. Amazon Quick Sight); a natural language processing service utilizing machine learning to determine insights and relationships in data, such as text data (e.g. Amazon Comprehend); a service for building, training, and/or deploying machine learning models (e.g. Amazon SageMaker); and a data warehouse service (e.g. Amazon Redshift).

Notably, the system 100, the process flow 200 and/or the platform 300 may conduct data collection, data capture, and analytics to gain valuable and life-saving insights in the fight against veteran and military suicide. Additionally, the system 100, the process flow 200 and/or the platform 300 may be utilized to establish a secure/HIPAA compliant data lake to collect and report on relevant data sets of the system 100, seek and ingest proprietary source of data available to the system 100, build a reporting capability to surface insights through reports, queries, dashboards, and visualizations, build the capacity to allow secure access to third party forensic organizations, utilize advanced machine learning and artificial intelligence techniques, and provide ongoing management of a dashboard including the various insights gleaned from the analyzed data. Based on the operation of the system 100, the system will have the data and initial research findings necessary to construct a hypothetical roadmap of the online footprint and digital activities of high-risk soldiers in the final moments of their lives. The system 100 may validate generated hypotheses through three independent processes. First, the system 100 may provide for real-time monitoring of online sites and social media platforms most frequented by service members and veterans (or other individuals) during the preparation and pre-action stages of suicidality with a focus on sites frequented in the last hour (or other period of time) of suicidal decedents' lives. Once the roadmap/online footprint for each individual is constructed by the system 100, the system 100 may employ strategic messaging to drive high-risk website visitors to the system 100 and/or to proxy sites generated by the system 100 for the purpose of risk triage. Once acquired, the system 100 will determine the effectiveness of specific monitoring, messaging and intervention strategies through suicide risk assessments for service members acquired through these methodologies. The model will demonstrate sensitivity and specificity for high-risk and low-risk levels. Second, the system 100 may identify the subset of active-duty clients assessed at highest suicide risk through the system's 100 evidence-based model demonstrating sensitivity and specificity for suicide risk. Once suicide risk is stabilized and managed, the system 100 investigators may work with each client to reconstruct his or her online activities in the last hours and days before they attempted suicide or reached out for emergency assistance. This process may be conducted via tools and methodologies approved as safe and effective by an independent evaluator of the system 100, with the advice and consultation of a Scientific Advisory Council of the system 100. Third, the system 100 may provide a smartphone application (or other application) for use with service member clients throughout the case management cycle for the purpose of monitoring and flagging emergent suicide risk between therapy sessions. This will enable the system 100 to contact case managers to intervene in real time to stabilize risk of suicide for an individual.

The system 100 may also facilitate and/or support suicide-prevention practices. For example, the system 100 may be utilized to establish and/or organize a series of on-site meetings and focus groups with a range of service member personnel. Once the meetings and focus groups are conducted, information associated with the meetings may be compiled and the system 100 may generate a report, which may include strategies for improving existing suicide prevention protocols and standard operating procedures. The system 100 may provide for routine installation-wide behavioral health check-ups for service members as well. The system 100 may also schedule and/or set up a series of installation site visits and USSOCOM unit reviews. Based on the reviews and visits, the system 100 may gather information associated with the review and visits to generate a final report of the findings and recommendations for improving army and USSOCOM suicide prevention programs, practices and tools. The system 100 may facilitate and/or provide resources, training and technical assistance to implement those recommendations approved by USSOCOM. The system 100 may also provide a structured training curriculum to ensure that the improvements are operationalized with fidelity and institutionalized.

The system 100 may support a service-delivery model that may begin with a semi-structured intake interview and comprehensive suicide risk assessment specifically developed for the system 100, such as by utilizing a Chronological Assessment of Suicide Events (CASE) approach. The assessment may involve engaging at-risk clients in a comprehensive dialogue about their military history, trauma, mental health, suicide attempt history, and recent thoughts or specific suicide plans, including those conditions most correlated with suicidal behavior. Simultaneously, providers utilizing the system 100 may build the connection and trust necessary to successfully reduce risk for at-risk individuals. After this assessment is complete, the system 100 and/or support professionals using the system 100 may classify at-risk clients as low, moderate, or high risk based on objective criteria and a review of risk factors. Following initial intake and assessment, social works of the system 100 may employ the Collaborative Assessment and Management of Suicidality (CAMS) approach, as modified for the system 100 to help at-risk clients learn how to manage their suicide risk and build resiliency over multiple therapy sessions. During the CAMS sessions, the system 100 and/or support professionals working with the system 100 may work with USSOCOM programs to link at-risk clients to needed resources and support at-risk clients in achieving higher level goals, such as sense of mission and greater connectedness to fellow soldiers. To disrupt the often-rapid descent from suicidal ideation to behavior, case managers utilizing the system 100 may work collaboratively with every at-risk client to develop individualized safety plans designed to make immediate access to highly lethal means more difficult during times of suicidal crises. The system 100 may also utilize Crisis Response Planning (CRP) safety planning protocols for all soldiers at risk for suicide. These safety plans may include strategies, such as safer storage practices (e.g. for storing weapons that may be utilized for self-destructive behavior), temporary handoff of weapons to buddies and family members, and creating “hope boxes” and similar methods of interrupting self-destructive behaviors.

The system 100 may also support an application for use with the clients and/or support professionals. For example, the application may be utilized to monitor at-risk clients and flag times of crisis (e.g. upon detection of suicidal behavior and/or self-destructive behavior and/or correlation with the models as described in the present disclosure) and provide immediate support for clients struggling with flare-ups in their suicidality until a case manager can personally respond. This service-delivery model provided by the system 100 may be adapted for remote delivery. In certain embodiments, functionality provided by the system 100 may involve use of telephones and supplemented video conference and text messaging to provide support to at-risk clients. All platforms of the system 100 supporting these functions may utilize encrypted messaging technology and may be HIPAA-compliant. The system 100 may also work with USSOCOM and key strategic partners, including Tricare, Cohen Veterans Network, Veteran and First Responder Healthcare and Centerstone, to provide tele-counseling and supportive services for at-risk clients with behavioral health needs. The adaptation for remote service provision by social workers under clinical supervision and the integration of these modalities into a seamless system 100 are key differentiators of the system's 100 service model, which has produced the following exemplary client outcomes unheard of in the field: Suicidality (−43%); Psychological Pain (−25%); Self-Hate (−26%); Agitation (−32%); Stress (−14%); and Hopelessness (−37%). These outcomes and multiple suicide risk metrics may be tracked longitudinally through the system's 100 data management system at 30-60-90-180-365-730 day (or other time) intervals, providing the longitudinal monitoring that holds such value for preventing, predicting, and treating suicidal behavior.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, accessing and/or extracting data from devices associated with deceased and/or living individuals; applying intelligence techniques to collect additional data associated with deceased and/or living individuals, such as from third-party data aggregators; processing data into a datasets; combining the datasets into a data lake; generating a model(s) associated with predictive behavior and patterns correlated with suicide based on an analysis and processing of the data; comparing behaviors and/or characteristics of living individuals to the model(s); identifying individuals at-risk for suicide and/or self-destructive behaviors based on the comparison; initiating services for identified individuals to reduce the risk of suicide and/or self-destructive behavior for the identified individuals; determining the effectiveness of the services in reducing suicide and/or self-destructive behaviors; and performing any other suitable operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

Although FIGS. 1-5 illustrates specific example configurations of the various components of the system 100, the system 100 may include any configuration of the components, which may include using a greater or lesser number of the components. For example, the system 100 is illustratively shown as including a first user device 102, a second user device 111, a communications network 135, a server 140, a server 145, a server 150, a server 160, and a database 155. However, the system 100 may include multiple first user devices 102, multiple second user devices 111, multiple communications networks 135, multiple servers 140, multiple servers 145, multiple servers 150, multiple servers 160, multiple databases 155, or any number of any of the other components inside or outside the system 100. Furthermore, in certain embodiments, substantial portions of the functionality and operations of the system 100 may be performed by other networks and systems that may be connected to system 100.

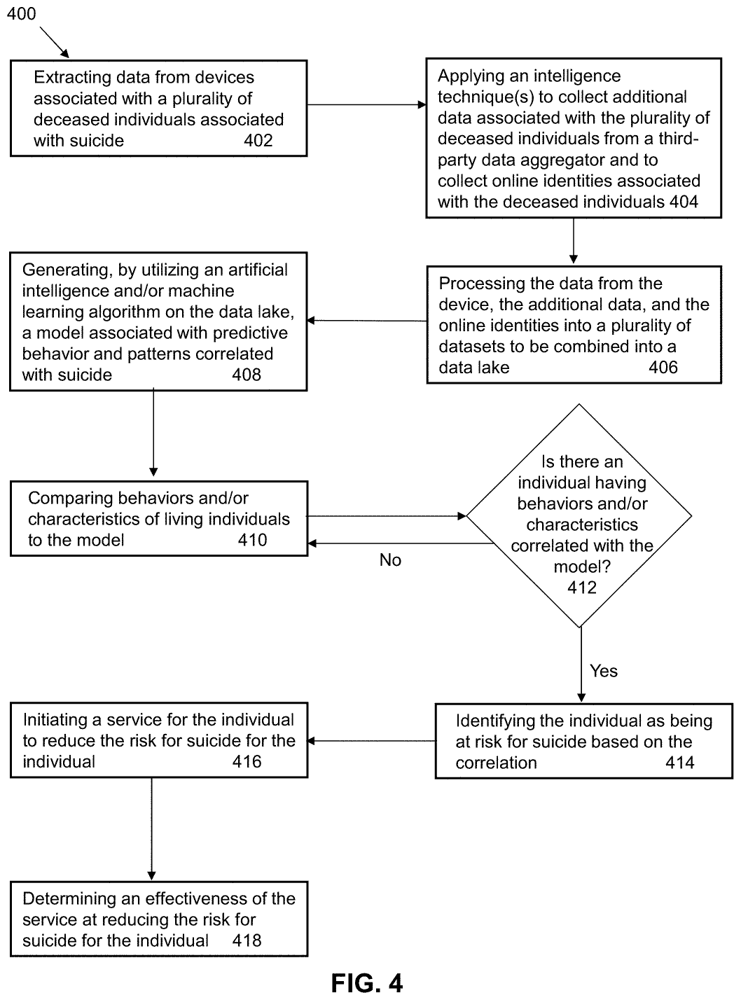

Notably, the system 100 may execute and/or conduct the functionality as described in the method(s) that follow. As shown in FIG. 4, an exemplary method 400 for utilizing digital forensics, artificial intelligence, and machine learning models to prevent suicidal behavior is schematically illustrated. The method 400 and/or functionality and features supporting the method 400 may be conducted via an application of the system 100, devices of the system 100, processes of the system 100, any component of the system 100, or a combination thereof. The method 400 may include steps for obtaining and analyzing data, processing the data into a data lake, generating models associated with predictive behavior and patterns associated with suicide and/or self-destructive behavior, identifying individuals correlated with one or more of the models, and initiating services to reducing the risk for suicide of identified individuals. At step 402, the method 400 may include accessing and/or extracting data associated with deceased individuals, such as those that have died via suicide, from devices associated with such individuals. For example, the method 400 may include extracting data from personal computers, smartwatches, mobile devices, sensors, laptops, tablets, and/or any other devices. The data may include, but is not limited to, social media posts, direct messages, information associated with contacts of the deceased individuals, search history, website history, blog posts, forum posts, mobile application activity, text messaging (SMS/MMS/other messaging forms), photos, videos, audio, media content, documents, files, downloaded information, uploaded information, video game profiles, video game history, patterns of usage relating to use of the devices, sensor data (e.g. heart-rate data, sweat data, oxygen data, biometric data, and/or any other sensor data), any other data, or a combination thereof. In certain embodiments, at step 402, the method 400 may also gather data from devices associated with living individuals as well. In certain embodiments, the accessing and/or extracting of the data from the devices may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device.

At step 404, the method 400 may include utilizing one or more intelligence techniques to collect additional data from third-party data aggregators that have data associated with the deceased individuals. The third-party data aggregators, for example, may be entities that have collected information associated with the deceased individuals from disparate online resources (and/or potentially offline resources), such as, but not limited to, websites, social media networks, surveys, subscriptions, user accounts, any type of online resource, or a combination thereof. In certain embodiments, the third-party data aggregators may be entities that do not have a direct relationship with the deceased individuals (and/or living individuals). Additionally, at step 404, the method 400 may also include collecting online identities and associated information of the deceased individuals (and/or living individuals) as well. In certain embodiments, OSINT techniques may be utilized to perform the data collections from the third-party data aggregators. In certain embodiments, the collecting of the additional data and/or online identities may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device. At step 406, the method 400 may include processing the data obtained from steps 402 and/or 404 into any number of datasets, which may be combined together into a data lake. The data lake may store raw forms of the data, disparate data, modified data, and/or any type of data. In certain embodiments, the processing of the data may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device.

At step 408, the method 400 may include utilizing artificial intelligence and/or machine learning algorithms and/or entity extraction applications against the data lake to generate one or more models of predictive behavior and patterns that are correlated with suicide and/or self-destructive behavior. A model may be a file, program, module, and/or process that may be trained by the system 100 to recognize certain patterns, behaviors, and/or content correlated with suicide and/or self-destructive behavior. The system 100 may train the model to reason and learn from data fed into the system 100 so that the model may generate and/or facilitate the generation of predictions about new data and information that enters into the system 100. As an example, as data is analyzed and/or processed by the system 100, certain images, websites, search history, sensor data, and/or other information may be determined by the system 100 to be associated with risk of suicide and/or self-destructive behavior. Such data may be tagged or otherwise marked as being associated with risk of suicide and/or self-destructive behavior for the model such that when new data is analyzed by the system 100, the system 100 may utilize the model's tagged information to determine whether the new data is associated with risk of suicide and/or self-destructive behavior. As an exemplary use-case scenario, if the model includes information indicating that if individuals access websites associated with suicide and such access of such websites is correlated with suicidal behavior and/or self-destructive behavior, then access of other websites having content that has a correlation with such websites may also be indicators of possible suicide and/or self-destructive behavior. In certain embodiments, each model and/or the information associated with each model may have a certain correlation, such as a numerical correlation, with suicide and/or self-destructive behavior. In certain embodiments, the higher the numerical correlation, the higher risk of suicide and/or self-destructive behavior the model may be associated with. In certain embodiments, the use of the artificial intelligence and/or machine learning algorithms and/or entity extraction applications against the data lake to generate the models may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device.

At step 410, the method 400 may include monitoring a group of living individuals and comparing behaviors and/or characteristics of the living individuals to the models correlated with suicide and/or self-destructive behavior. In certain embodiments, the monitoring and/or comparing may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device. At step 412, the method 400 may include determining if there is any individual(s) having behaviors and/or characteristics correlated with the model(s). For example, the system 100 may monitor the behavior and/or characteristics of an individual and determine that content accessed on a website by the individual correlates with the model as being associated with a threshold amount of suicide risk and/or self-destructive behavior. In certain embodiments, the determining may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device. If, at step 412, the method 400 does not determine that there are any individuals having behaviors and/or characteristics correlated with the model, the method 400 may revert back to step 410 and continue with the steps of the method 400. If, however, at step 412, the method 400 determines that there is an individual or individuals having behaviors and/or characteristics correlated with the model, the method 400 may proceed to step 414. At step 414, the method 400 may include identifying the individual(s) from the group of monitored individuals that have the behaviors and/or characteristics correlated with the model(s) as being at risk for suicide and/or self-destructive behavior based on their correlation with the model. In certain embodiments, the identifying may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device.

Once the individual(s) are identified as being at risk for suicide and/or for self-destructive behavior, the method 400 may proceed to step 416, which may include initiating one or more services for the individual(s) to reduce the risk for suicide and/or the self-destructive behavior. For example, the system 100 may cause a signal to be sent to a device of the user that interrupts the user from continuing the self-destructive behavior and/or a suicide attempt. The signal may cause the individual to be redirected to a suicide prevention website, to receive audio and/or media content to persuade the individual from the behavior, to receive a link to initiate a digital therapy session with a medical expert and/or therapist, to receive a link to schedule a therapy session with the individual, to receive alternative media content in place of existing media content and/or online resources that the individual is currently interacting with, to receive a digital questionnaire to obtain information associated with the individuals' state of mind, to receive any type of interruption and/or service, or a combination thereof. In certain embodiments, the service may be initiated via an application on a device of the individual, via a text message link sent via a text message to the device of the individual, via an instant message, via a phone call, via any digital means, or a combination thereof. In certain embodiments, the service may be initiated by sending a signal to a device of a person associated with the individual, such as a family member, friend, and/or acquaintance. In certain embodiments, the initiating of the one or more services may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device.

Once one or more services are initiated and/or rendered to the individuals at risk for suicidal behavior and/or self-destructive behavior, the method 400 may proceed to step 418, which may include determining an effectiveness of the service at reducing the risk for suicide and/or self-destructive behavior for the individual. For example, the effectiveness may be determined via a survey with the at-risk individual, by determining that the individual has changed their behavior based on further monitoring of the individual, by receiving confirmation from the individual such as by phone call, digital message, etc., by receiving a confirmation from an individual associated with the at-risk individual, by determining that the behaviors and/or characteristics no longer have a correlation with the models of the system 100, by receiving a confirmation from a health expert who works with the individual, by any other means, or a combination thereof. In certain embodiments, the determining of the effectiveness may be performed and/or facilitated by utilizing the first user 101, the second user 110 and/or by utilizing the first user device 102, the second user device 111, the server 140, the server 145, the server 150, the server 160, the communications network 135, any combination thereof, or by utilizing any other appropriate program, network, system, or device. Notably, the method 400 may further incorporate any of the features and functionality described for the system 100, any other method disclosed herein, or as otherwise described herein.

The systems and methods disclosed herein may include still further functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and the various methods discloses herein. For example, by training the system 100 over time based on data and/or other information provided and/or generated in the system 100, a reduced amount of computer operations may need to be performed by the devices in the system 100 using the processors and memories of the system 100 than compared to traditional methodologies. In such a context, less processing power needs to be utilized because the processors and memories do not need to be dedicated for processing. As a result, there are substantial savings in the usage of computer resources by utilizing the software, techniques, and algorithms provided in the present disclosure. In certain embodiments, various operative functionality of the system 100 may be configured to execute on one or more graphics processors and/or application specific integrated processors.

Notably, in certain embodiments, various functions and features of the system 100 and methods may operate without any human intervention and may be conducted entirely by computing devices. In certain embodiments, for example, numerous computing devices may interact with devices of the system 100 to provide the functionality supported by the system 100. Additionally, in certain embodiments, the computing devices of the system 100 may operate continuously and without human intervention to reduce the possibility of errors being introduced into the system 100. In certain embodiments, the system 100 and methods may also provide effective computing resource management by utilizing the features and functions described in the present disclosure. For example, in certain embodiments, devices in the system 100 may transmit signals indicating that only a specific quantity of computer processor resources (e.g. processor clock cycles, processor speed, etc.) may be devoted to generating the predictive models associated with suicide, identifying individuals having a correlation with the generated models, initiating services to reduce the risk for suicide for identified individuals, and/or performing any other operation conducted by the system 100, or any combination thereof. For example, the signal may indicate a number of processor cycles of a processor may be utilized to generate predictive models, and/or specify a selected amount of processing power that may be dedicated to generating or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected value. In certain embodiments, the system 100 and methods may include transmitting signals to the memory devices utilized in the system 100, which indicate which specific sections of the memory should be utilized to store any of the data utilized or generated by the system 100. Notably, the signals transmitted to the processors and memories may be utilized to optimize the usage of computing resources while executing the operations conducted by the system 100. As a result, such functionality provides substantial operational efficiencies and improvements over existing technologies.

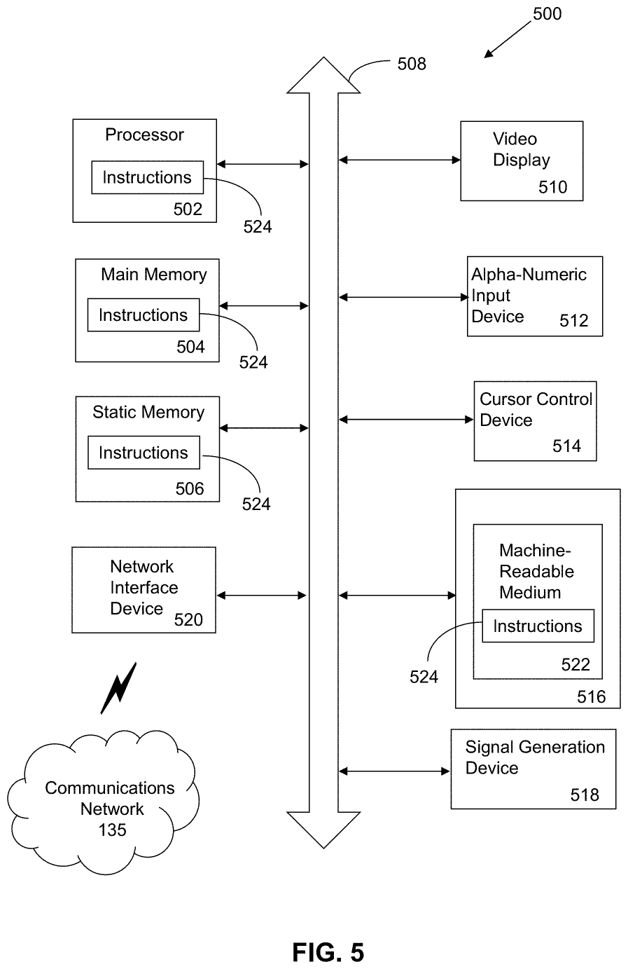

Referring now also to FIG. 5, at least a portion of the methodologies and techniques described with respect to the exemplary embodiments of the system 100 can incorporate a machine, such as, but not limited to, computer system 500, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100. As another example, the computer system 500 may assist with generating models associated with predictive behaviors and/or patterns associated with suicide. As yet another example, the computer system 500 may assist with identifying individuals having behaviors and/or characteristic having a correlation with the generated models, and then initiating services to reduce the risk of suicide for such identified individuals.

In some embodiments, the machine may operate as a standalone device. In some embodiments, the machine may be connected (e.g., using communications network 135, another network, or a combination thereof) to and assist with operations performed by other machines and systems, such as, but not limited to, the first user device 102, the second user device 111, the server 140, the server 145, the server 150, the database 155, the server 160, any other system, program, and/or device, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 500 may include a processor 502 (e.g., a central processing unit (CPU), a graphics processing unit (GPU, or both), a main memory 504 and a static memory 506, which communicate with each other via a bus 508. The computer system 500 may further include a video display unit 510, which may be, but is not limited to, a liquid crystal display (LCD), a flat panel, a solid state display, or a cathode ray tube (CRT). The computer system 500 may include an input device 512, such as, but not limited to, a keyboard, a cursor control device 514, such as, but not limited to, a mouse, a disk drive unit 516, a signal generation device 518, such as, but not limited to, a speaker or remote control, and a network interface device 520.

The disk drive unit 516 may include a machine-readable medium 522 on which is stored one or more sets of instructions 524, such as, but not limited to, software embodying any one or more of the methodologies or functions described herein, including those methods illustrated above. The instructions 524 may also reside, completely or at least partially, within the main memory 504, the static memory 506, or within the processor 502, or a combination thereof, during execution thereof by the computer system 500. The main memory 504 and the processor 502 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 522 containing instructions 524 so that a device connected to the communications network 135, another network, or a combination thereof, can send or receive voice, video or data, and communicate over the communications network 135, another network, or a combination thereof, using the instructions. The instructions 524 may further be transmitted or received over the communications network 135, another network, or a combination thereof, via the network interface device 520.

While the machine-readable medium 522 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor the executes the instructions to perform operations, the operations comprising:
extracting data from devices associated with a plurality of deceased individuals associated with suicide;
applying an intelligence technique to collect additional data associated with the plurality of deceased individuals from a third-party data aggregator and to collect online identities associated with the plurality of deceased individuals;
processing the data from the devices, the additional data, and the online identities into a plurality of datasets to be combined into a data lake;
generating, by utilizing an artificial intelligence algorithm on the data lake, a model associated with predictive behavior and patterns correlated with suicide;
identifying an individual at risk for suicide based on the individual having a behavior, characteristic, or both, correlated with the model; and
initiating a service for the individual to reduce the risk for suicide for the individual.

2. The system of claim 1, wherein the operations further comprise validating a predictor of suicidal behavior by utilizing the model.

3. The system of claim 1, wherein the data comprises a social media post, a direct message, online account information, search history, website history, blog information, forum information, activity conducted on a mobile application, activity conducted on a device, a text message, media content associated with the deceased individuals, documents associated with the deceased individuals, gaming profiles and history associated with the deceased individuals, or a combination thereof.

4. The system of claim 1, wherein the operations further comprise constructing a hypothetical digital roadmap of an online footprint for the individual by utilizing the model.

5. The system of claim 1, wherein the service comprises transmitting a message to a device of the individual directing the individual to a suicide prevention tool, monitoring the individual for suicide-correlated behavior, conducting an intervention for the individual, contacting a therapist or physician to assist the individual, transmitting an alert to the device of the individual, conducting a digital interview with the individual, or a combination thereof.

6. The system of claim 1, wherein the operations further comprise updating the model as further data from the devices, further additional data from the third-party data aggregator, further online identities, or a combination thereof, are obtained over time.

7. The system of claim 1, wherein the operations further comprise generating deceased user profiles for the plurality of deceased individuals, wherein the deceased user profiles include the data from the devices, the additional data, the online identities, the model, information associated with the behavior, information associated with the service, or a combination thereof.

8. The system of claim 7, wherein the operations further comprise identifying the individual at risk for suicide based on a user profile of the individual having a correlation with any of the deceased user profiles having a threshold risk associated with suicide.

9. The system of claim 1, wherein the operations further comprise monitoring an online site, a social media platform, a mobile application, or a combination thereof, utilized by the individual at risk during a preparation stage or pre-action stage of suicidality.

10. The system of claim 1, wherein the operations further comprise facilitating reconstruction of activities conducted by the individual at risk for a selected period of time prior to a suicide attempt or an emergency contact made by the individual at risk.

11. The system of claim 1, wherein the operations further comprise facilitating disruption of the behavior by utilizing the service.

12. The system of claim 1, wherein the operations further comprise determining a situational trigger for triggering the individual at risk to contemplate suicide.

13. The system of claim 1, wherein the operations further comprise determining a factor associated with increased risk of suicide based on an analysis of the data from the devices, the additional data, the online identities, the model, information associated with the behavior, information associated with the service, or a combination thereof.

14. A method, comprising:
extracting data from devices associated with a plurality of deceased individuals associated with suicide;
applying an intelligence technique to collect additional data associated with the plurality of deceased individuals from a third-party data aggregator and to collect online identities associated with the plurality of deceased individuals;
processing the data from the devices, the additional data, and the online identities into a plurality of datasets to be combined into a data lake;
generating, by utilizing instructions from a memory that are execute by a processor and by utilizing an artificial intelligence algorithm on the data lake, a model associated with predictive behavior and patterns correlated with suicide;
identifying an individual at risk for suicide based on the individual having a behavior, characteristic, or both, correlated with the model; and
initiating a service for the individual to reduce the risk for suicide for the individual.

15. The method of claim 14, further comprising generating a report, a query, a digital dashboard, a visualization, or a combination thereof, including information associated with the data from the devices, the additional data, the online identities, the model, information associated with the behavior, information associated with the service, or a combination thereof.

16. The method of claim 14, further comprising facilitating, for the individual at risk, reduction of a risk factor associated with suicide.

17. The method of claim 14, further comprising analyzing a group of individuals at risk for suicide, and further comprising identifying a subset of individuals from the group of individuals that have a threshold suicide risk based on a comparison to the model.

18. The method of claim 17, further comprising initiating services for the subset of individuals from the group of individuals that have the threshold suicide risk to reduce a probability of suicide.

19. The method of claim 14, further comprising determining an effectiveness of the service in reducing the risk for suicide for the individual.

20. A computer-readable device comprising instructions, which, when loaded and executed by a processor, cause the processor to perform operations, the operations comprising:
extracting data from devices associated with a plurality of deceased individuals associated with suicide;
applying an intelligence technique to collect additional data associated with the plurality of deceased individuals from a third-party data aggregator and to collect online identities associated with the plurality of deceased individuals;
processing the data from the devices, the additional data, and the online identities into a plurality of datasets to be combined into a data lake;
generating, by utilizing an artificial intelligence algorithm on the data lake, a model associated with predictive behavior and patterns correlated with suicide;
identifying an individual at risk for suicide based on the individual having a behavior, characteristic, or both, correlated with the model; and
initiating a service for the individual to reduce the risk for suicide for the individual.
